(19) United States
(12) Patent Application Publication
(10) Pub. No.: US 2011/0245604 A1
(43) Pub. Date: Oct. 6, 2011

(54) CAPSULE ENDOSCOPE AND CAPSULE ENDOSCOPY SYSTEM

(76) Inventors: CHUEN-TAI YEH, Hsinchu City (TW); Tah-Yeong Lin, Longtan Township (TW); Chia-Sung Wu, Taoyuan City (TW); Yu-Te Chen, Guanxi Township (TW)

(21) Appl. No.: 12/750,704

(22) Filed: Mar. 30, 2010

(51) Int. Cl. A61B 1/045; A61B 1/00

(52) U.S. Cl. 600/109; 600/118

(57) ABSTRACT

The present invention provides a capsule endoscope and a capsule endoscopy system. The capsule endoscope includes a power supply module, an image function module and a power control module. The image function module is configured to capture the image outside the capsule endoscope. The power control module is electrically connected to the power supply module and includes an electronic switch. The power control module controls the electronic switch according to a predetermined condition to control the electric power provided from the power supply module to the image function module. By this configuration, the power control module is able to suspend the power supply until the capsule endoscope reaches the specific location in the digestive tract, and then starts the power supplying to enable the successive image inspection of the capsule endoscope. Therefore, the electric power is conserved to enable the inspection of the end portion of digestive system of the capsule endoscope.

### BACKGROUND OF THE INVENTION

1. Field of the Invention

The present invention relates generally to a capsule endoscope and a system thereof, and more particularly, to a capsule endoscope and a system thereof of which the power can be activated remotely.

2. Description of the Prior Art

The risk that modern people suffer from digestive cancers, such as colorectal cancer, has risen with the changing lifestyle and diet habits. If the inspection of the digestive tract can be performed regularly, then the cancer may be detected at its initial stage and be treated to reduce the risk of death caused by it. Inspection on digestive tract/system/organs nowadays is generally carried out by the endoscope. By inserting an endoscope with a camera head thereon into the digestive tract through the mouth or anus, the image of the digestive tract can be captured in real time to perform the inspection.

Most of the endoscopes are designed in such a way that the illumination is provided by a light source from an external machine. Lights from the light source will be guided by the optical fiber to the camera to illuminate the vicinity of the camera. Furthermore, according to different types of camera, the captured optical images or digital images need to be transmitted to the external system by the optical fiber or electronic circuits.

Therefore, a catheter is needed to be placed between the camera and external system to accommodate the optical fiber or electronic circuits.

In the practical application, the inspectors will insert the endoscope catheter deep into the digestive tract through mouth or anus of a patient. When progressing through the digestive tract, the captured image from the camera at the front side of the catheter can be displayed on a screen of the external system. However, because the digestive of the human body is twisted and turned, the catheter will inevitably rub, collide or drag the digestive tract. The above-mentioned situation will result in tremendous pain to the patients, which may scare the patient away from the inspection, and therefore delays the treatment from the best treatment timing.

Furthermore, the catheter of the conventional endoscope can only reach the front end of the small intestine from the mouth or the rear-end of the small intestine form the anus. Because the small intestine of the human can be up to 6 meters long with twists and turns, the catheter is not able to move along the small intestine accordingly and therefore is not able to perform complete and detailed inspection of the small intestine.

To solve this problem, the capsule endoscope system is then developed. The capsule endoscope system comprises an external system and a capsule endoscope. The capsule endoscope comprises an illumination module, an image sensing module, a wireless transmitting module and a battery, which are all integrated into a small capsule of about 2.6 cm in length and 1.1 cm in diameter. The capsule endoscope can be swallowed by the patient and move along the digestive tract with the help of peristalsis of the digestive tract. The illumination module illuminates the surrounding of the capsule by the LEDs so that the image sensing module can capture the image along the digestive tract. The captured image is then able to be transmitted to the external system by the wireless transmitting module. The external system can store the image so that the inspector can view the image recorded in real time or later.

By performing the gastrointestinal endoscopy with the capsule endoscope, it can prevent the patient from suffering the pain brought by the conventional endoscope while being able to capture clear images of the small intestine. However, because the volume of the capsule endoscope is limited to the condition that it has to be small enough to be swallowed while large enough to accommodate multiple elements, the capacity of the equipped battery is then limited to the volume thereof. Generally, the capacity of the battery of the capsule endoscope is about 50 mAh, which can only supply the capsule endoscope to work for about 8 hours with regard to the currently electronic technology. In practical application, when the capsule endoscope passes the small intestine and reaches the large intestine, the power thereof is often already exhausted and unable to continue with the work. Therefore, when performing the gastrointestinal inspection of the end part of the digestive tract, it still relies on the conventional endoscope and can't benefit from the capsule endoscopy.

### SUMMARY OF THE INVENTION

The objective of the present invention is to provide a capsule endoscope to solve the problems in the prior art.

According to one embodiment of the present invention, the capsule endoscope comprises a power supply module, an image function module and a power control module. The image function module is electrically connected to the power supply module and configured to capture an image outside the capsule endoscope. The power control module is electrically connected to the power supply module and comprises an electronic switch. The power control module switches the electronic switch according to a predetermined condition so as to control the power output to the image function module from the power supply module.

Another objective of the present invention is to provide a capsule endoscope system to solve the problems in the prior art.

According to one embodiment of the present invention, the capsule endoscope system comprises an external control system and a capsule endoscope. The external control system comprises a remote control module, which is adapted to transmit a remote power control signal wirelessly. The capsule endoscope comprises a power supply module, an image function module and a power control module. The image function module is configured to capture an image outside the capsule endoscope and to transmit the image to the external system. The power control module is electrically connected to the power supply module and comprises an electronic switch. The power control module switches the electronic switch according to the remote power control signal so as to control the power outputted to the image function module from the power supply module.

In conclusion, the capsule endoscope according to the present invention is able to switch the electronic switch according to a predetermined condition and therefore is able to determine to supply the power to the image function module of the capsule endoscope or not. By this configuration, the capsule endoscope is able to shut down the image capturing function before the capsule endoscope reached the area of interest so that the power can be reserved for the inspection of the end part of the digestive tract. The predetermined condition can be a signal triggered from a timer after a predetermined time or triggered wireles sly by a signal transmitted from the external control system and therefore grants considerable operating flexibility in practical application.

The advantage and spirit of the invention may be understood by the following recitations together with the appended drawings.

### BRIEF DESCRIPTION OF THE APPENDED DRAWINGS

FIG. 1 is a schematic view of the capsule endoscope system according to the first embodiment of the present invention.

FIG. 2 is a functional block diagram of the capsule endoscope system according to the first embodiment of the present invention.

FIG. 3 is a functional block diagram of the capsule endoscope system according to the second embodiment of the present invention.

FIG. 4 is a functional block diagram of the capsule endoscope system according to the third embodiment of the present invention.

### DETAILED DESCRIPTION OF THE INVENTION

The present invention provides a capsule endoscope and a system thereof to capture the image of the large intestine wall. The embodiments of the present invention will be described in the following detailed description.

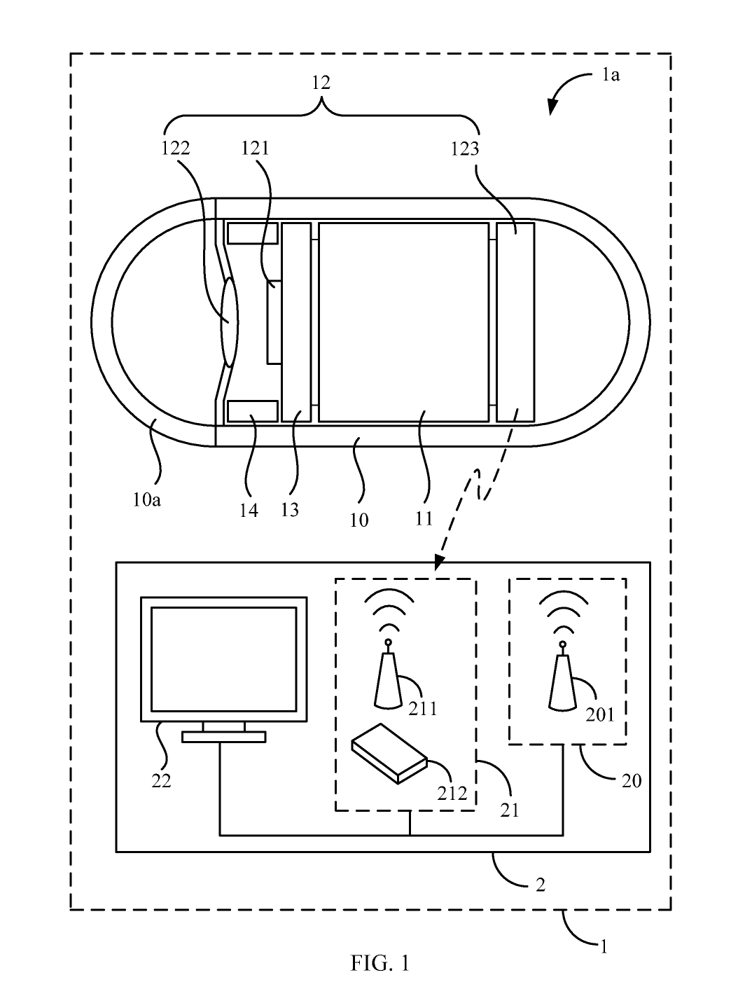

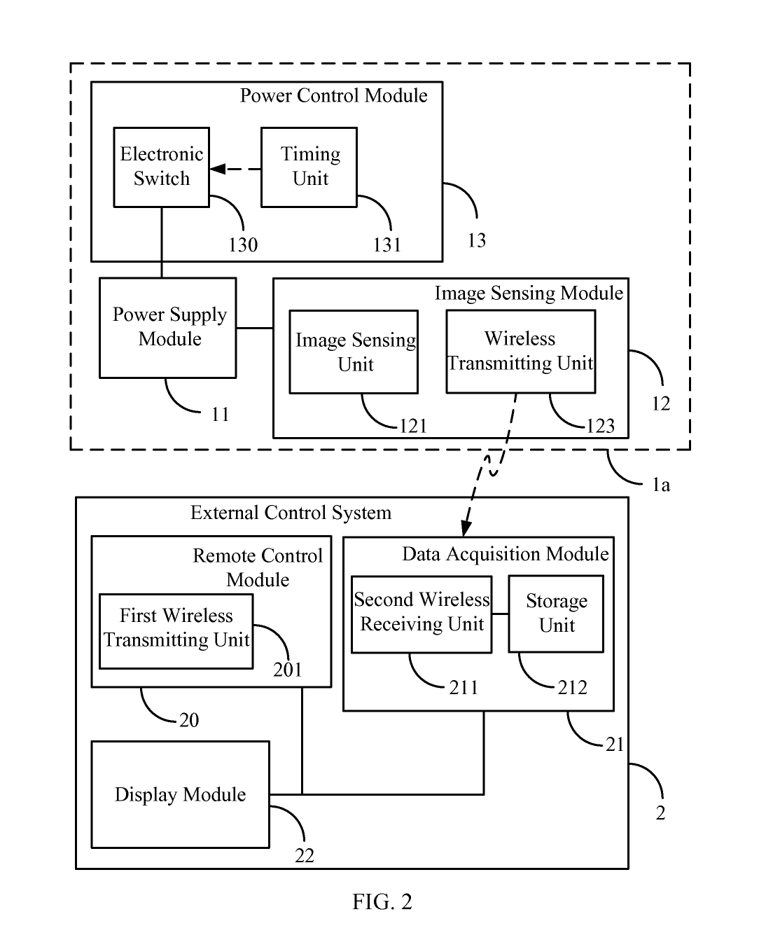

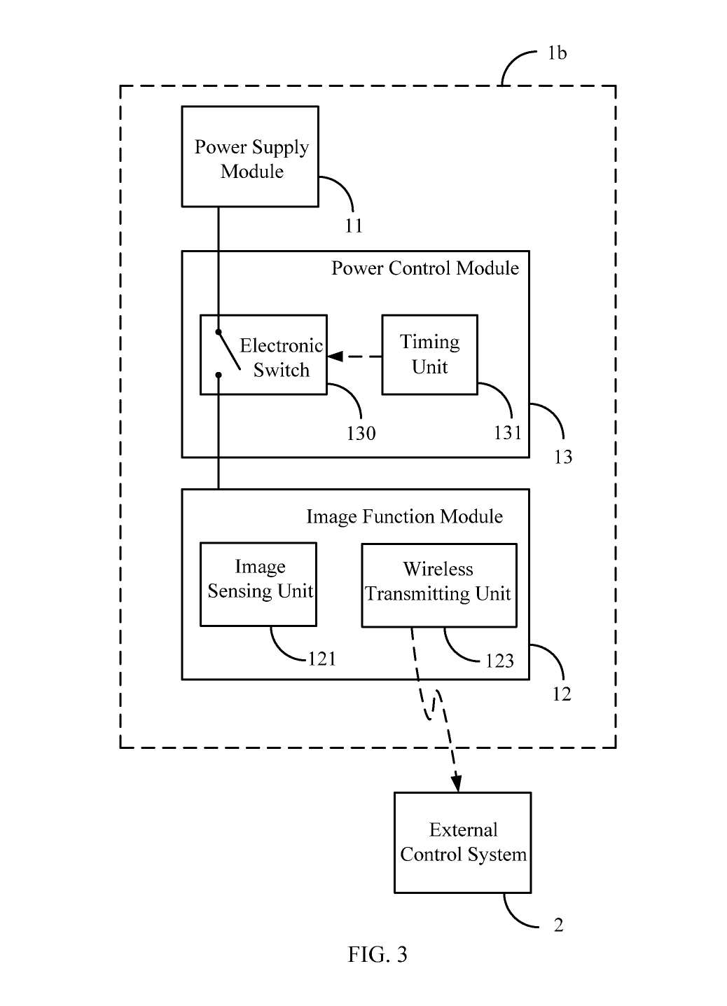

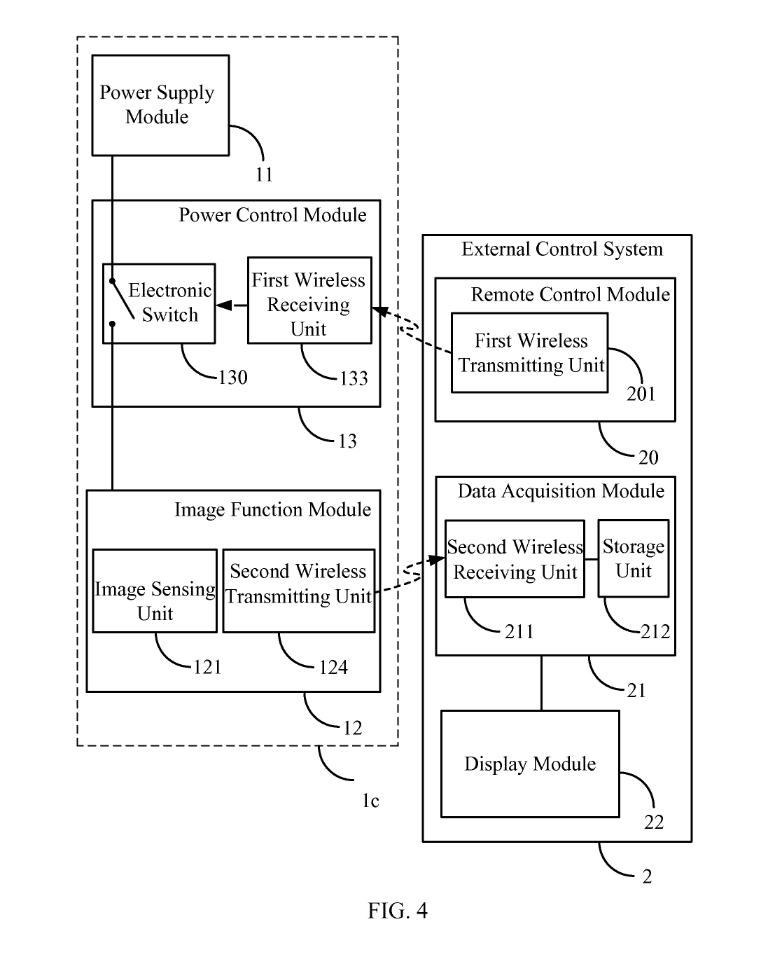

FIG. 1 is a schematic view of the capsule endoscope system of the first embodiment according to the present invention. FIG. 2 is a functional block diagram of the capsule endoscope system of the first embodiment according to the present invention. FIG. 3 is a functional block diagram of the second embodiment according to the present invention. FIG. 4 is a functional block diagram of the third embodiment of the present invention.

Referring to FIGS. 1 and 2 together, the capsule endoscope system 1 of the first embodiment according to the present invention is depicted. The capsule endoscope system 1 comprises the capsule endoscope 1a and the external control system 2. The capsule endoscope 1a can be swallowed into the digestive tract of a human body and interact with the external control system 2. The external control system 2 comprises the remote control module 20, the data acquisition module 21 and the display module 22. The remote control module 20 comprises a first wireless transmitting unit 201 and is able to transmit a control signal to the capsule endoscope 1a by the first wireless transmitting unit 201. The data acquisition module 21 comprises a second wireless receiving unit 211 and a storage unit 212 for receiving and storing the data transmitted by the capsule endoscope 1a respectively. The data acquisition module 21 can be an independent device, which can be equipped on the patient to receive the data transmitted from the capsule endoscope 1a at anytime. The data received from the capsule endoscope 1a by the data acquisition module 21 can be sent to the display module 22 for the inspection in real time or later.

The capsule endoscope 1a comprises the capsule shell 10, the power supply module 11, the image function module 12, the power control module 13 and the light emitting diodes 14. The power supply module 11 is a battery, and is disposed inside the capsule shell 10 together with the image function module 12, the power control module 13 and the light emitting diodes 14. The front side of the capsule shell 10 comprises a transparent dome 10a, which allows the image outside the capsule shell 10 to be projected onto the image function module 12 and allows the light emitting diodes 14 to emit the light outward the capsule shell 10.

The image function module 12 is electrically connected to the power supply module 11 and configured to capture an image outside the capsule endoscope 1a. The image function module 12 comprises image sensing unit 121 and a lens 122. The image sensing unit 121 comprises a photoelectric sensor and a processing circuit. The photoelectric sensor may be a Complementary Metal-Oxide-Semiconductor (CMOS) or a Charge-Couple device (CCD). The image outside the capsule shell 10 enters the capsule shell 10 through the transparent dome 10a and is then focused on to the photoelectric sensor of the image sensing unit 121 by the lens 122. Following, the processing circuit will convert the electrical signal on the photoelectric sensor into a digital data. The acquired digital data can be stored in an additional memory disposed inside the capsule endoscope 1a. Alternatively, the image function module 12 may further comprises a wireless transmitting unit 123 to communicate to the external control system 2 so as to transmit the digital data to the external control system 2.

The power control module 13 is electrically connected to the power supply module 11 and comprises the electronic switch 130. The power control module 13 switches the electronic switch 130 according to a predetermined condition to control the operation of the power supply module 11, so as to further control the power output to the image function module 12. More specifically, the electronic switch 130 determines whether the state of the power supply module 11 is to be activated or turned off. In this configuration, even if the direct electrical connection exists between the power supply module 11 and the image function module 12, no power will be transmitted through the electric circuit between the power supply module 11 and the image function module 12 in the condition that the power supply module 11 is shut down by the electronic switch 130. In this embodiment, the power control module 13 further comprises a timing unit 131 and is configured to take a predetermined delay time as the predetermined condition in coordination with the timing unit 131. Because the power control module 13 has to maintain the state of time counting, the timing unit 131 is selected from the elements of lower power consumption under the consideration of power consuming. For example, the micro-power comparator, the operation amplifier or a micro-power processor can be operated to perform time counting function with the power consumption of less than 1 mA. Furthermore, the CMOS can be served as the power control module 13 to further reduce the power consumption since the internal resistance of CMOS is generally less than 1 ohm. To facilitate the manufacturing, the circuit of the power control module 13 can be disposed on the same circuit board of the image sensing unit 121 or integrate into the same chip of the image sensing unit 121.

For example, if the capsule endoscope 1a is desired to capture the image of the patient's large intestine, the predetermined delay time of the power control module 13 can be set to 8 hours after the capsule endoscope 1a being swallowed by the patient. The power control module 13 will determine whether to switch the electronic switch 130 on or off according to the counting of timing unit 131. Before the time passes 8 hours, the power control module 13 will switch off the electronic switch 130 so that the power supply module 11 won't supply the power to the image function module 12, which saves the power of the capsule endoscope 1a. The capsule endoscope 1a will approximately reach the large intestine 8 hours after being swallowed. At the same time, the power control module 13 will determine that the time has passed the predetermined 8 hours according to the timing unit 131 and therefore switch on the electronic switch 130 so that the power supply module 11 will supply the power to the image function module 12. In such a way, the capsule endoscope 1a is able to initiate image capturing when it reaches the large intestine.

FIG. 3 shows the capsule endoscope system 1 of the second embodiment according to the present invention which comprises the capsule endoscope 1b and the external control system 2. In this embodiment, the power control module 13 of the capsule endoscope 1b is connected in series between the power supply module 11 and the image function module 12. More specifically, the electronic switch 130 of the power control module 13 is serial connected on the circuit, through which the power supply module 11 supplies the power to the image function module 12, and therefore determines whether the circuit is opened or closed between the power supply module 11 and the image function module 12. By this configuration, the circuit design of the capsule endoscope 1b can be simplified.

FIG. 4 shows the capsule endoscope system 1 of the third embodiment of the present invention which comprises the capsule endoscope 1c and the external control system 2. The external control system 2 comprises a remote control module 20, the second wireless receiving unit 211 and the display module 22. The remote control module 20 comprises a first wireless transmitting unit 201. The capsule endoscope 1c comprises the power supply module 11, the image function module 12 and the power control module 13. The image function module 12 comprises the image sensing unit 121 and the second wireless transmitting unit 124. The power control module 13 comprises the electronic switch 130 and the first wireless receiving unit 133. The external control system 2 transmits the remote power control signal by the first wireless transmitting unit 201. The remote power control signal will then be received by the first wireless receiving unit 133. The content of the remote power control signal can be the order whether to switch on or off the electronic switch 130. The power control module 13 will then further switch the electronic switch 130 according to the remote power control signal.

More specifically, after the capsule endoscope 1c being swallowed into the digestive tract, the inspector can operate the external control system 2 at anytime to send a remote power control signal from the first wireless transmitting unit 201 of the remote control module 20. If the content of the remote power control signal is to switch on the electronic switch 130 or the image function module 12, then the power control module 13 will switch on the electronic switch 130 so that the power supply module 11 can supply the power to the image function module 12. After the image function module 12 is activated, the image sensing unit 121 will capture the image outside the capsule endoscope 1c and convert the captured image into the digital data. The digital data will then be transmitted to the external control system by the second wireless transmitting unit 124. Next, the display module 22 will display a real-time image around the capsule endoscope 1c according to the received digital data. Before the capsule endoscope 1c reaches the area to be inspected, the inspector can send an order of switching off the electronic switch 130 or shutting down the image function module 12 by the first wireless transmitting unit 201 so as to stop the power supply to the image function module 12 from the power supply module 11.

By using the capsule endoscope system 1 of the third embodiment, the inspector can remotely control the power supplying to the capsule endoscope 1c before the capsule endoscope 1c reaches the large intestine. The capsule endoscope 1c will be activated to perform the inspection only at appropriate time and then be disabled to reserve the power of the battery. In such a way, not only the progressing speed of the capsule endoscope 1c in the digestive tract can be traced but also the capsule endoscope 1c is able to reserve enough power for the inspection of large intestine wall after it reaches the large intestine. Furthermore, the transmission of the remote power control signal and the digital data of the image utilize different band of the radio wave or magnetic field and therefore will not interfere with each other.

In conclusion, by using the capsule endoscope system of the present invention, the time to perform the image capturing and transmission function of the capsule endoscope can be determined by the countdown of the delay time or the remote control. With such configuration, the power consumption can be reduced and reserved for the capsule endoscope to extend the inspection range to the large intestine. In view that the lesions of the large intestine occur more than in the small intestine, the technique of the present invention is in need to facilitate the application of the capsule endoscope able to be swallowed applying to the inspection of large intestine.

With the example and explanations above, the features and spirits of the invention will be hopefully well described. Those skilled in the art will readily observe that numerous modifications and alterations of the device may be made while retaining the teaching of the invention. Accordingly, the above disclosure should be construed as limited only by the metes and bounds of the appended claims.

What is claimed is:

1. A capsule endoscope, comprising:
a power supply module;
an image function module, configured to capture an image outside the capsule endoscope; and
a power control module, electrically connected to the power supply module and comprising an electronic switch, the power control module switching the electronic switch according to a predetermined condition so as to control a power output to the image function module from the power supply module.

2. The capsule endoscope of claim 1, wherein the power control module comprises a timing unit and is configured to switch on the electronic switch after a predetermined delay time according to the timing unit so as to activate the image function module.

3. The capsule endoscope of claim 2, wherein the timing module is a micro-power comparator, an operational amplifier or a micro-power processor.

4. The capsule endoscope of claim 1, wherein the power control module comprises a wireless receiving unit, the wireless receiving unit is configured to receive a remote power control signal from an external control system, and the power control module switches the electronic switch according to the remote power control signal.

5. The capsule endoscope of claim 1, wherein the electronic switch is a MOSFET and is serial connected between the power supply module and the power control module.

6. The capsule endoscope of claim 1, wherein the image function module is further configured to transmit the image to an external control system.

7. The capsule endoscope of claim 6, wherein the image function module comprises an image sensor unit and a wireless transmitting unit, wherein the image sensor unit is configured to convert the image outside the capsule endoscope into a digital data, and the wireless transmitting unit is configured to transmit the digital data to the external control system.

8. A capsule endoscope system, comprising:
an external control system, comprising a remote control module adapted to transmit a remote power control signal; and
a capsule endoscope, comprising a power supply module, an image function module and a power control module;
wherein the image function module is configured to capture an image outside the capsule endoscope and to transmit the image to the external control system, the power control module is electrically connected to the power supply module and comprising an electronic switch, the power control module switches the electronic switch according to the remote power control signal so as to control a power output to the image function module from the power supply module.

9. The capsule endoscope system of claim 8, wherein the remote control module comprises a first wireless transmitting unit, the power control module comprises a first wireless receiving unit, the image function module comprises a second wireless transmitting unit, the external control system further comprises a second wireless receiving unit, the remote power control signal is transmitted to the first wireless receiving unit from the first wireless transmitting unit, and the image is transmitted to the second wireless receiving unit from the second wireless transmitting unit.

10. The capsule endoscope system of claim 9, where in the external control system further comprises a display module configured to display the image received by the second wireless receiving unit.
